(19) United States
(12) Patent Application Publication
(10) Pub. No.: US 2005/0165315 A1
(43) Pub. Date: Jul. 28, 2005

(54) Side firing fiber optic array probe

(75) Inventors: Andres F. Zuluaga, Boston, MA (US); Jeffrey A. Korn, Lexington, MA (US)

Correspondence Address: HOUSTON ELISEEVA, 4 MILITIA DRIVE, SUITE 4, LEXINGTON, MA, 02421, US

(73) Assignee: InfraReDx, Inc., Cambridge, MA (US)

(21) Appl. No.: 10/765,626

(22) Filed: Jan. 27, 2004

(51) Int. Cl. A61B 6/00

(52) U.S. Cl. 600/476; 600/473; 600/478

(57) ABSTRACT

A multi fiber optic medical probe comprises at least two optical fibers. There are side-firing terminations for the at least two optical fibers. Further, beam-shaping apertures are provided for controlling light propagating between the side-firing terminations and a region lateral to the probe. The provision of the at least two optical fibers allows for multiple optical signals to be transmitted to and/or from the target area within the patient. The side-firing terminations allow for the interrogation of regions that are adjacent to the probe, i.e., extending in a direction parallel to the insertion direction or longitudinal axis of the probe. The beam shaping apertures are provided for controlling light propagating between the side-firing terminations and the region lateral to the probe, in order to control the shape of the emitted beam and also, the direction from which light is collected.

### BACKGROUND OF THE INVENTION

Probe-based, such as catheter-based, optical systems are applicable to a number of diagnostic and therapeutic medical applications. Optical coherence tomography is used to provide spatial resolution, enabling the imaging of internal structures. Spectroscopy is used to characterize the composition of structures, enabling the diagnosis of medical conditions by differentiating between cancerous, dysplastic, and normal tissue structures, for example. Ablation systems are used to remove or destroy structures within the body to address various diseases, such as tachycardias, tumors, and coronary artery disease, in another example of a probe-based optical system.

For example, in one specific spectroscopic application, an optical source, such as a tunable laser, is used to access or scan a spectral band of interest, such as a scan band in the near infrared wavelengths or 750 nanometers (nm) to 2.5 micrometers (μm). The generated light is used to illuminate tissue in a target area in vivo using the catheter. Diffusely reflected light resulting from the illumination is then collected and transmitted to a detector system, where a spectral response is resolved. The response is used to assess the composition and consequently the state of the tissue.

This system can be used to diagnosis atherosclerosis, and specifically to identify atherosclerotic lesions or plaques. This is an arterial disorder involving the intimae of medium- or large-sized arteries, including the aortic, carotid, coronary, and cerebral arteries.

Diagnostic systems including Raman and fluorescence-based schemes have also been proposed. Other wavelengths, such as visible or the ultraviolet, can also be used.

The probes or catheters for these applications typically have small lateral dimensions. This characteristic allows them to be inserted into incisions or lumen, such as blood vessels, with lower impact or trauma to the patient. The probe's primary function is to convey light to and/or receive light from a target area or area of interest in the patient. In the context of the diagnosis of atherosclerosis, for example, the target areas are regions of the patient's arteries that may exhibit or are at risk for developing atherosclerotic lesions.

For many of these applications, the target areas or areas of interest are located lateral to the probe. That is, in the example of lumens, the probe is advanced through the lumen under analysis until it reaches the areas of interest, which are typically the lumen walls that are adjacent to the probe, i.e., extending parallel to the direction of advance of the probe.

In these applications, “side-firing” probes are used. These probes emit and/or receive light from along the probe's lateral sides. In the example of light emission, the light propagates through the probe, until it reaches the probe or catheter head. The light is then redirected to be emitted radially or in a direction that is orthogonal to the direction of advancement of the probe. In the case of light collection, light from along the probe's lateral sides is collected and then transmitted through the probe to an analyzer where, in the example of spectroscopic analysis in the diagnosis of atherosclerosis, the spectrum of the returning light is resolved in order to determine the composition of the vessel or lumen walls.

In the case of very small gauge devices, angle polishing is typically used to create the side-firing probe. In these examples, the probe or catheter is manufactured from optical fiber. The terminal end of optical fiber is then angle polished so that light propagating down the optical fiber is reflected by the reflective, angular endface to be emitted in a radial direction to a region that is lateral to the probe. In the opposite example of light collection, the angle polished head reflects light directed radially at the probe from regions lateral to the probe to be coupled into the optical fiber's core to be transmitted to the analyzer.

In these applications, the problem of astigmatism has been addressed. Specifically, in the example of side-firing probes fabricated from angle polished fibers, the emitted beam will typically be astigmatic without further beam correcting structures. This is due to the lensing effect of the fiber's curved sidewalls.

Solutions to this astigmatism problem have been proposed. Some have compensated for the curvature of the lateral side of the fiber by adding other beam controlling surfaces. Others have proposed to remove the curvature by polishing.

### SUMMARY OF THE INVENTION

The manufacture or assembly of these conventional side-firing probes is costly and a time-consuming, however. Moreover, they are typically poorly adapted for multi-fiber probes. These are commonly required, for example, in spectroscopic applications, where one fiber carries light to the target area or area of interest, and then one or more other fibers are used to collect the light from the target area for further analysis. In these spectroscopic applications, it is also sometimes important to control the separation between the emitted beam and the region from where the light is collected. Moreover, different types of fiber are often used to transmit the light to the target area as opposed to collect light from it.

In general, according to one aspect, the invention features a multi fiber optic medical probe. The probe comprises at least two optical fibers. There are side-firing terminations for the at least two optical fibers. Further, beam-shaping apertures are provided for controlling light propagating between the side-firing terminations and a region lateral to the probe.

The provision of the at least two optical fibers allows for multiple optical signals to be transmitted to and/from the target area within the patient. The side-firing terminations allow for the interrogation of regions that are adjacent or lateral to the probe. The beam shaping apertures are provided for controlling light propagating between the side-firing terminations and the region lateral to the probe, in order to control the shape of the emitted beam and also, the direction from which light is collected.

In one embodiment, the at least two optical fibers comprise just two optical fibers. However, in other embodiments, the at least two optical fibers comprise eight or more separate optical fibers.

In the preferred embodiment, the two optical fibers comprise at least one single mode fiber and at least one multi-mode fiber. For example, in the context of the single mode fiber, the core diameter of the optical fiber is usually less than about 10 micrometers, whereas the core diameter of the multi-mode fiber is usually greater than 100 micrometers. Typically, the single mode fiber is used to transmit light to the target area and the multi-mode fiber is used to collect light from the target area.

In the preferred embodiment, the side-firing terminations comprise angled endfaces for the at least two optical fibers. These angled endfaces are preferably formed by polishing. Reflectivity is achieved by the refractive index mismatch between the fiber and air, for example. In other examples, however, the endfaces are metal coated to provide the required reflectivity. In still further examples, multilayer dielectric thin film coatings are used to form the mirrors.

In another embodiment, the side-firing terminations comprise at least one coreless block. This coreless block preferably comprises an angled endface, which can be formed by polishing and metal coated, in one example. The coreless block typically has an index of refraction that is similar to the fiber. It does not have a light guiding core, however. The coreless block is typically attached to a cleaved end of the optical fiber. It is fused to those optical fibers, in one example.

In the preferred embodiment, at least one capillary tube is provided over the side-firing terminations of the at least two optical fibers. The at least one capillary tube provides the beam shaping apertures. In one example, a single capillary tube is used with multiple bores for receiving each of the optical fibers. In another example, a separate capillary tube is placed over each of the optical fibers. The capillary tubes then attach, such as bonded to each other.

An advantage of this embodiment is that it provides a non-astigmatic design and enables rigid alignment between the fibers, leveraging existing connector processes. Assembly is easy, due to the flat, controlled surfaces at the catheter's distal tip, provided by the capillary tubes. It is easy to adjust the separation, and fixturing is also expedited.

In still further embodiments, spacers can be provided between the capillary tubes. Further, a wedge spacer can be used for controlling the angle between the optical axes of the beam shaping apertures for each of the optical fibers. This wedge spacer can be integral with one of the capillary tubes and formed, such as by polishing.

In still other embodiments, the beam shaping apertures are longitudinally offset along an axis of the probe, with respect to each other. This is another way of controlling the distance between the optical axes of one of the beam shaping apertures with respect to another one of the beam shaping apertures.

In general, according to another aspect, the invention also features a method for gathering optical information, using a medical probe. This method comprises transmitting an optical signal in a first optical fiber, and then directing the optical signal radially to a region lateral to the probe, with a side-firing termination. The beam shape of the optical signal is controlled. Finally, optical information is collected with a second optical fiber. The optical information is then transmitted to an analyzer.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIGS. 1A and 1B are a perspective view and a bottom plan view of a multi fiber optic medical probe, according to a first embodiment of the present invention;

FIG. 2 is a perspective view of a second embodiment of the inventive multi fiber optic medical probe;

FIGS. 3A and 3B are a side plan view and a perspective view of the side-firing termination and beam shaping aperture for one of the optical fibers;

FIG. 4 is a perspective view of a third embodiment of the multi fiber optic medical probe, comprising four optical fibers;

FIG. 5 is a perspective view of a fourth embodiment of the multi-fiber optical medical probe using coreless blocks for the side-firing terminations and beam shaping apertures;

FIG. 6 is a fifth embodiment of the multi fiber optic medical probe using coreless blocks and longitudinally off-set beam shaping apertures;

FIGS. 7A-7D are plan, end views of four different embodiments, illustrating ways of controlling the lateral and angular separation between the optical axes of the beam shaping apertures, according to the present invention;

FIG. 8 is a perspective view of the capillary tubes used in embodiments of the present invention;

FIGS. 9A and 9B are a top plan view and a side plan view of an eight fiber optic medical probe, according to a sixth embodiment of the present invention, which also illustrate the manufacture of the probe;

FIG. 10A is a schematic block diagram illustrating a catheter-based medical optical system, to which the inventive multi-fiber optic medical probes are applicable; and

FIG. 10B is a cross-sectional view oft he probe head positioned adjacent tissue, illustrating the operation of the multi-fiber optic probe, according to the present invention.

### DETAILED DESCRIPTION OF THE PREFERRED EMBODIMENTS

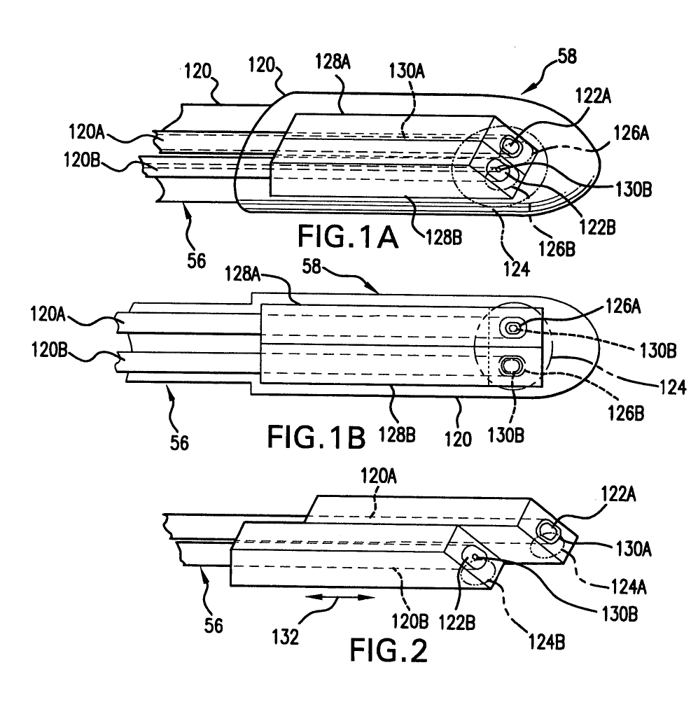

FIGS. 1A and 1B show the terminal end of a multi fiber optic medical probe, which has been constructed according to the principles of the present invention.

In more detail, the probe or catheter head 58 comprises an outer casing 120. In some examples, this outer casing 120 is transmissive to the optical signals of interest or the wavelength ranges of light of interest. In other examples, the casing 120 is generally non-transmissive, but has a transmissive window structure.

Multiple optical fibers extend through the catheter 56 to the catheter's head 58, within the casing 120. In this first embodiment, the two optical fibers 120A, 120B are provided. The terminal ends of these optical fibers 120A, 120B have side-firing terminations 122A, 122B. In one embodiment, these side-firing terminations 122A, 122B are coated to reflect light. In other examples, the index mismatch between the material of the optical fibers 120A, 120B and the medium adjacent to the side-firing terminations 122A, 122B, such as air, provides the required reflectivity. The side-firing terminations 122A, 122B, in effect, couple the optical fibers 120A, 120B to a region 124 that is lateral to the probe head 58.

Specifically, light emanating from the region 124 and directed radially with respect to an toward the fibers is reflected by the side-firing terminations 122A, 122B to be coupled into and propagated by the optical fibers 120A, 120B. Similarly, light propagating through the optical fibers 120A, 120B to the side-firing terminations 122A, 122B is reflected to be directed radially to the region 124, which is lateral to the probe head 58.

One problem that arises, however, with these side-firing terminations for optical fibers is astigmatism in the emitted beam or the collected light, due to the propagation of the light through the curved side walls of the optical fibers 120A, 120B. In the preferred embodiment, beam shaping apertures 126A, 126B are provided for controlling the light propagating between the side-firing terminations 120A, 120B and the region lateral to the probe head 58.

In the first embodiment of FIGS. 1A and 1B, the beam shaping apertures 126A, 126B are provided by square or rectangular cross-sectioned capillary tubes 128A, 128B. These are preferably inserted over the optical fibers 120A, 120B. They are further preferably bonded to the optical fibers 120A, 120B, using an epoxy or other bonding material that is preferably index-matched to the material of the optical fibers 120A, 120B. Therefore, light traveling between the side-firing terminations 122A, 122B and the lateral region 124, does not have any astigmatic lensing since the light does not “see” the curved sidewalls of the fibers 120A, 120B.

In the preferred embodiment, the optical fibers 120A, 120B have light guiding cores 130A, 130B to transmit the light along the longitudinal length of the catheter 56 with low loss.

In the current embodiment, a combination of multi-mode fiber and single transverse mode fiber is used. Specifically, in the illustrated embodiment, optical fiber 128A is a multi-mode optical fiber, i.e., the fiber supports multiple transverse modes of the wavelength used by the system, which is typically in the infrared wavelengths. Specifically, the fiber's core 130A is large. Preferably, it is larger than 100 micrometers in diameter. This allows it to efficiently collect light from the lateral region 124 and transmit it down the optical fiber 128A.

In contrast, optical fiber 120B is preferably single mode fiber. Specifically, the diameter of the optical fiber's core 130B is preferably less than 10-15 micrometers i.e., the fiber supports only a single transverse mode efficiently of the wavelength used by the system, which is typically in the infrared wavelengths. This allows it to couple to a single mode light source and then, transmit that light to the lateral region 124, with a predictable Gaussian distribution.

FIG. 2 shows a second embodiment of the multi-fiber optic medical probe. In this example, the side-firing terminations 122A and 122B of optical fibers 120A and 120B, respectively, are longitudinally offset with respect to each other. The side-firing terminations are located at different positions along the longitudinal axis 132 of the catheter 56. As a result, the optical fiber 120A collects light from region 124A, whereas optical fiber 120B emits light into region 124B. This embodiment has the advantage of being able to control the position where the light is emitted and collected. It allows increases in the path length of light traveling from optical fiber 120B to 120A without substantially increases the width of the probe.

In the embodiments illustrated in FIGS. 1A, 1B, and 2, the manufacturing process for the multi-fiber optic medical probe is as follows. First, any coating or sheath on the optical fibers 120A, 120B is stripped. The optical fibers 120A, 120B are then slid or inserted into the axial bores in their respective capillary tubes 128A, 128B. In one embodiment, quartz/fused silica capillary tubes are used. Then, an index-matched adhesive or epoxy is applied between the fibers 120A, 120B and the axial bores of their respective capillary tubes 128A, 128B. The refractive index of the epoxy is preferable matched to the refractive index of the cladding layer of the respective optical fibers 120A, 120B. The viscosity should further be selected such that it wicks into the small gap between the bore's wall of the capillary tubes 128A, 128B and the optical fibers 120A, 120B.

Further, adhesive with no spectral features in the wavelength window of the scan band should be selected.

The adhesive is then cured. Finally, the optical fibers 120A, 120B and their respective capillary tubes 128A, 128B are polished together. A coating can finally be added to increase the reflectivity of the side-firing terminations 122A, 122B.

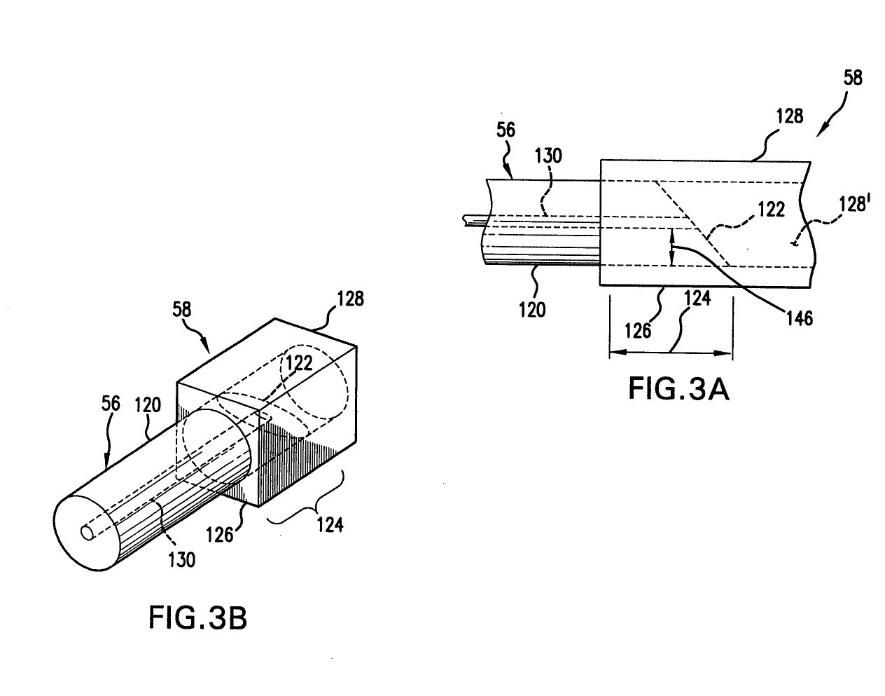

FIGS. 3A and 3B illustrate side-firing terminations and beam shaping apertures fabricated according to a different process. Here, the optical fiber's side-firing termination 122 is fabricated before insertion into the capillary tube. As before, the side-firing termination optically couples the optical fiber's core 130 to the region 124 that is adjacent to the side-firing probe head 58, along the side-firing optical axis 146. The optical fiber 120 is then inserted in a capillary tube 128. As a result, in this example, the side-firing termination 122 is recessed into the capillary tube 128. This has advantage in that, if the region 128′ is air filled, or filled with another low index material, the side-firing termination 122 will be inherently reflective even without any coating step, due to the index mismatch between the optical fiber's core 130 and the low index medium filling region 128′, such as air.

The manufacturing sequence for this embodiment is as follows. Again, the fiber coating is stripped. Then the terminal end of the optical fiber 120 is polished to form the side-firing termination 122. Optical fiber is then inserted into the capillary tube 128 and bonded to the tube such that any space between the fiber's side wall and the inner bore of the capillary tube 128 is filled with index matching epoxy material, especially along the optical axis stretching between the side-firing termination 122 and the beam shaping aperture 126.

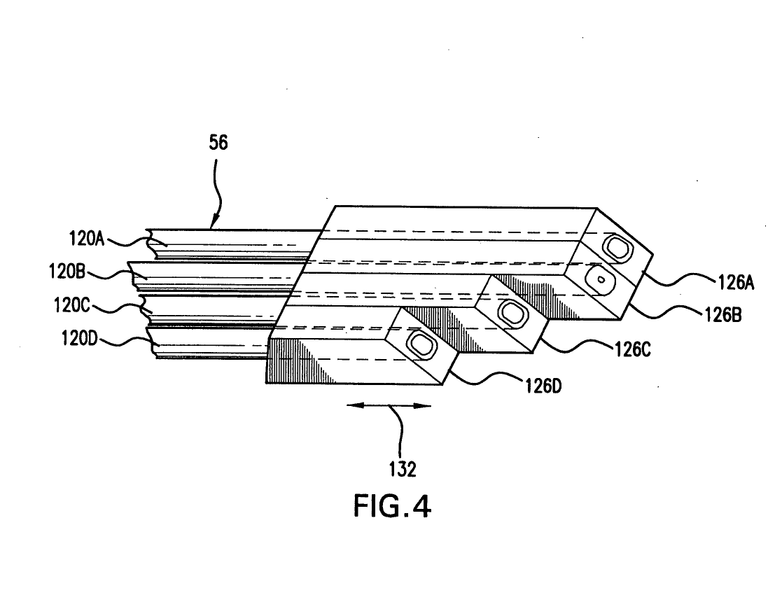

FIG. 4 shows a third embodiment of the multi-fiber optic medical probe. In this example, the four optical fibers 120A-128D are provided. Each has respective beam shaping apertures 126A-126D.

The advantage of the third embodiment is that multiple collection multimode optical fibers are provided. The multiple multi-mode fibers 126A, 126C, 126D are located at different longitudinal positions along the longitudinal axis 132 of the probe 56. This allows light emitted by the single mode optical fiber 120B to be collected at multiple distances by the multimode collection optical fibers 120A, 120C, 120D. This allows the spectral response to be collected from different pathlengths.

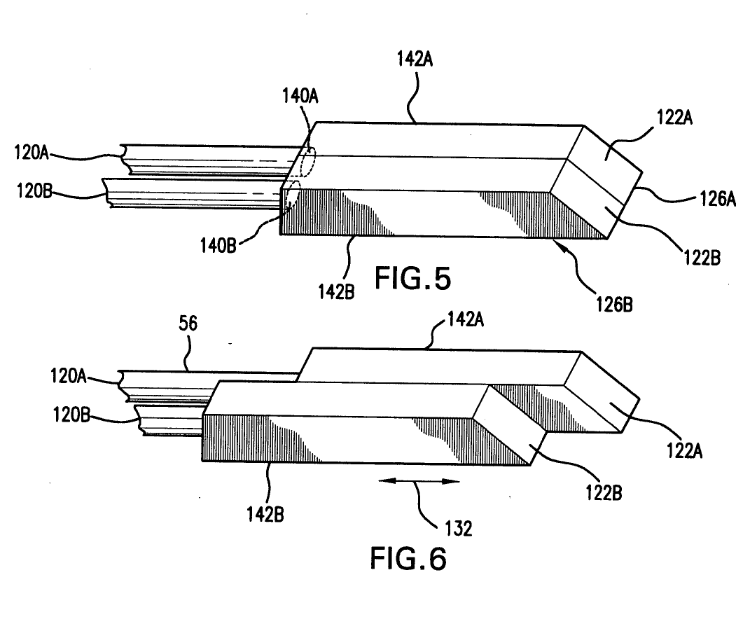

FIG. 5 shows a fourth embodiment of the inventive multi fiber optic medical probe. In this example, the optical fibers 120A, 120B are terminated, for example, in a flat cleave end 140A, 140B. These are attached to coreless blocks 142A, 142B. These blocks have square or rectangular cross-sections. Note that, although two coreless blocks are shown, in other embodiments, a single coreless block that has a rectangular cross section could be used.

In one embodiment, the optical fibers 120A, 120B are fused to their respective coreless blocks 142A, 142B. The coreless blocks have angled endfaces that provide the side-firing terminations 122A, 122B. The coreless blocks sidewalls provide the beam shaping apertures 126A, 126B. The reflectivity of these side-firing terminations 122A, 122B can be enhanced with a metal coating or dielectric thin film coatings as described previously.

FIG. 6 shows a fifth embodiment where the side-firing terminations 122A, 122B are longitudinally offset along the axis 132 of the catheter 56 to thereby control the distance between the lateral regions to which they are coupled.

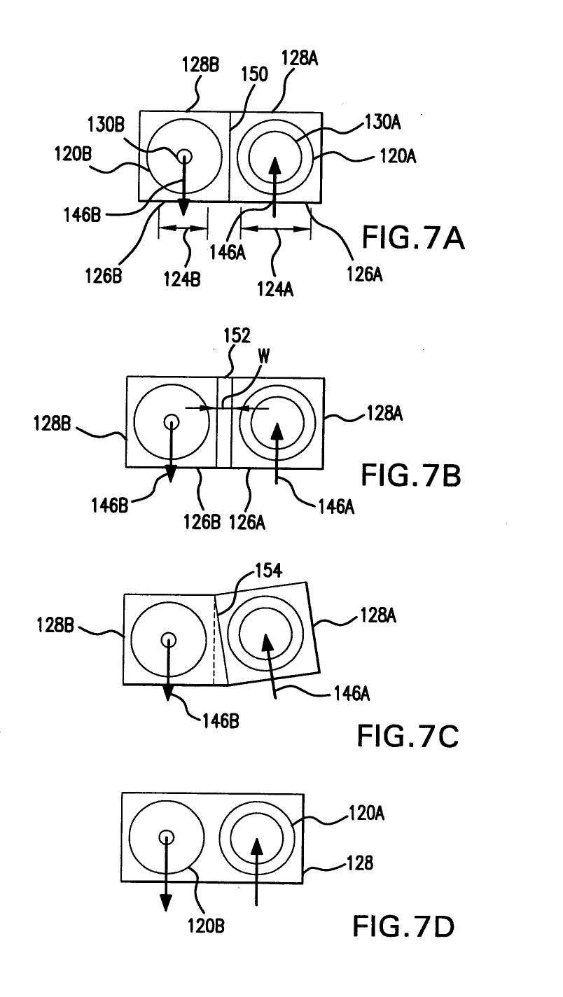

FIGS. 7A-7D illustrate various configurations for controlling the separation and angle between the optical axes 146A, 146B of the side-firing terminations of optical fibers 120A, 120B. Specifically, the optical axes 146A, 146B are defined as being orthogonal to the beam shaping surfaces 126A, 126B or radial to the fiber 120.

In the basic example, illustrated in FIG. 7A, the axes of beam shaping surfaces 126A, 126B are parallel to each other. Further, the capillary tubes 128A, 128B are simply bonded to each other along the interface 150.

In the embodiment illustrated in FIG. 7B, the lateral separation between the optical axes 146B, 146A is increased by including a spacer block 152 between the capillary tubes 128A and 128B.

The width W of this spacer block 152 is used to control the separation between the optical axes 146A, 146B defined by the beam shaping apertures 126B, 126A.

In the embodiment of FIG. 7C, a wedge spacer block 154 is used. In a typical embodiment, the wedge spacer block 154 is integral with one of the capillary tubes 128. Specifically, it can be fabricated by angle polishing a side of one of the capillary tubes 128. The wedge block 52 is used to increase the separation between optical axes 146A, 146B. It is also used here to adjust the angular separation between the optical axes, such that they are either converging toward each other or diverging, as illustrated in the embodiment in FIG. 7C.

FIG. 7D shows still another embodiment, in which a single capillary tube 128 is used. The capillary tube, however, has multiple bores for the optical fibers 120A, 120B.

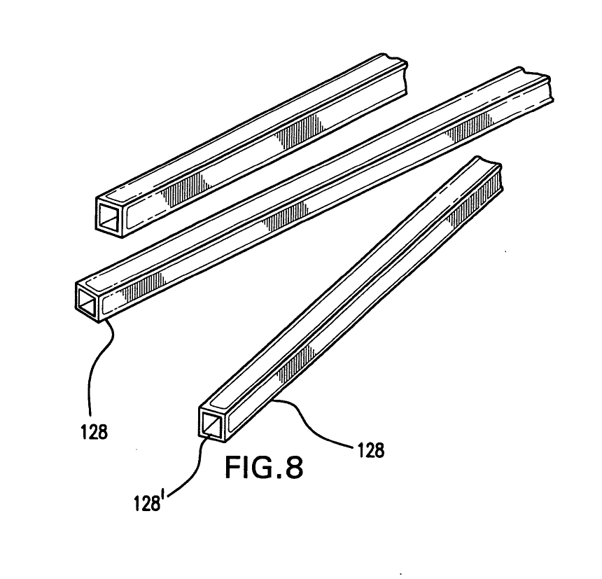

FIG. 8 shows a number of examples of the capillary tubes 128 that are used in the fabrication of the present invention. Here, they are shown without the optical fibers 120 inserted into their central, axial bores 128′. In one example, the capillary tubes 128 are fabricated by drawing a borosilicate glass preform.

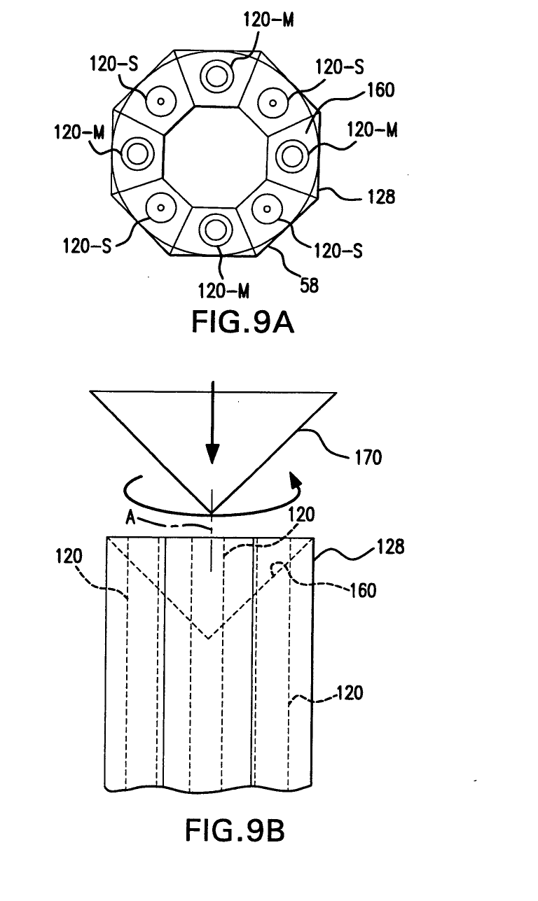

FIGS. 9A and 9B illustrate a sixth embodiment of the multi fiber optic probe 58. Specifically, an octagonal capillary tube 128 is provided. This can either be comprised of a single orthogonal capillary tube 128 that has been drawn. Or, as illustrated, multiple capillary tubes are assembled to form the octagonal cross-section of the probe head 58. Multiple bores are provided into which a series of optical fibers 120 are inserted. In the illustrated example, alternating single mode fibers 120S and multi-mode fibers 128M are provided circumferentially around the periphery of the capillary 128. A conical blind hole 160 is then formed into the end of the octagonal capillary tube 128.

The conical bore 160 is fabricated as illustrated in FIG. 9B, in one implementation. Specifically, a conical abrasive polishing element 170 is inserted down the center axis A of the capillary tube 128. This forms the side-firing terminations for each of the optical fibers 120 in the capillary or composite capillary 128.

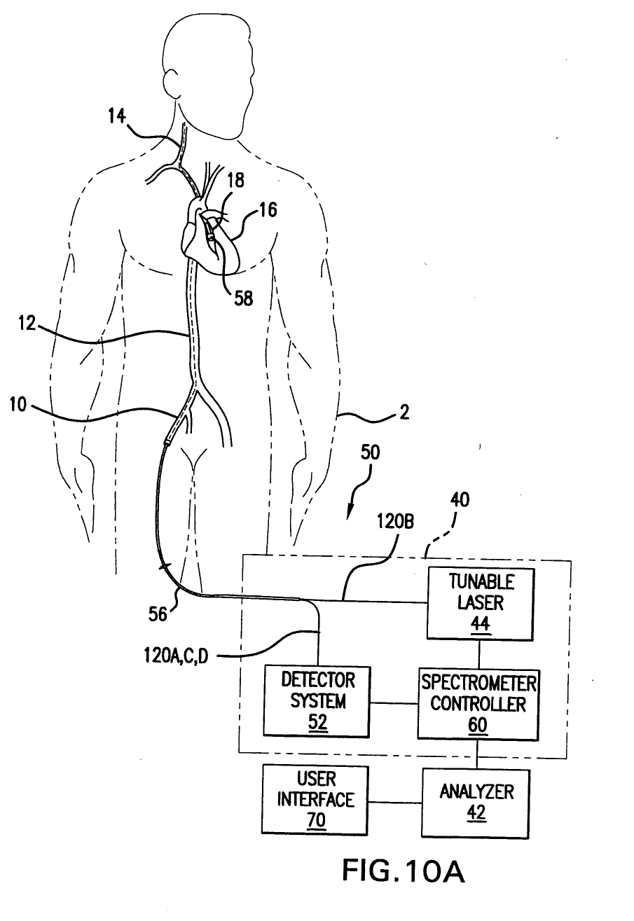

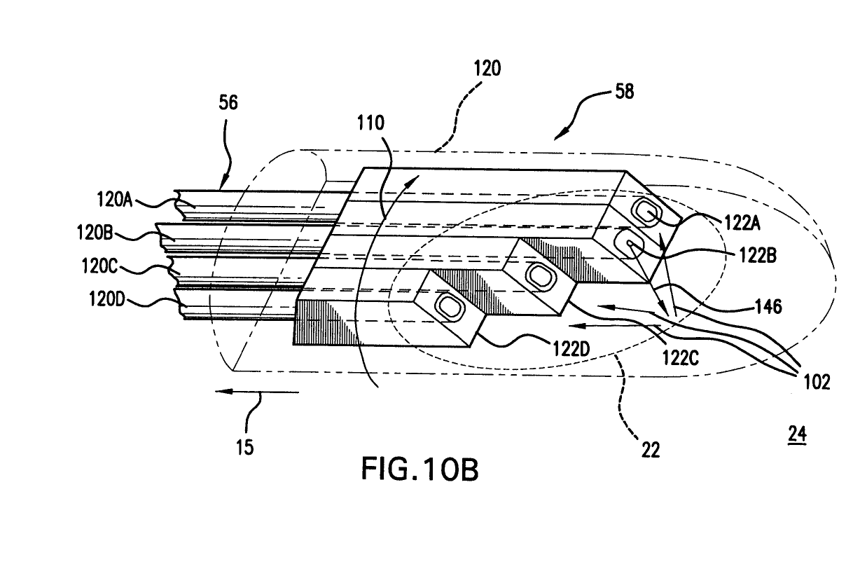

FIG. 10A shows an optical spectroscopic catheter system 50 for blood vessel analysis, to which the present invention is applicable, in one example.

The system generally comprises the probe, such as, catheter 56, a spectrometer 40, and analyzer 42. In many cases, the catheter rides on a guide wire that is first advanced through the patient's blood vessels.

In more detail, the catheter 56 includes the optical fiber bundle. The catheter 56 is typically inserted into the patient 2 via a peripheral vessel, such as the femoral artery 10. The catheter head 58 is then moved to a desired target area, such as a coronary artery 18 of the heart 16 or the carotid artery 14. In the embodiment, this is achieved by moving the catheter head 58 up through the aorta 12.

When at the desired site, radiation is generated. In the current embodiment optical radiation is generated, preferably by a tunable laser source 44 and tuned over a range covering one or more spectral bands of interest. In other embodiments, one or more broadband sources are used to access the spectral bands of interest. In either case, the optical signals are coupled into the single mode fibers 120-B of the catheter 56 to be transmitted to the catheter head 58.

In the current embodiment, optical radiation in the near infrared (NIR) spectral regions is used for spectroscopy. Exemplary scan bands include 1000 to 1450 nanometers (nm) generally, or 1000 nm to 1350 nm, 1150 nm to 1250 nm, 1175 nm to 1280 nm, and 1190 nm to 1250 nm, more specifically. Other exemplary scan bands include 1660 nm to 1740 nm, and 1630 nm to 1800 nm.

However, in other optical implementations, scan bands appropriate for fluorescence and/or Raman spectroscopy are used. In still other implementations, scan bands in the visible or ultraviolet regions are selected.

In the current embodiment, the returning light is transmitted back down the multimode optical fibers 120-A, C, D of the catheter 56. The returning radiation is provided to a detector system 52, which can comprise one or multiple detectors.

A spectrometer controller 60 monitors the response of the detector system 52, while controlling the source or tunable laser 44 in order to probe the spectral response of a target area, typically on an inner wall of a blood vessel and through the intervening blood or other unwanted signal source, which is typically a fluid.

As a result, the spectrometer controller 60 is able to collect spectra. When the acquisition of the spectra is complete, the spectrometer controller 60 then provides the data to the analyzer 42.

With reference to FIG. 10B, the optical signal along the optical axis 146 from the optical fiber of the catheter 56 is directed by the side firing termination 122B, to exit from the catheter head 58 and impinge on the target area 22 of the artery wall 24. The catheter head 58 then collects the light that has been diffusely reflected or refracted (scattered) from the target area 22 and the intervening fluid and returns the light 102 back down the catheter 56 through the multimode fibers 120-A, C, D.

In one embodiment, the catheter head 58 spins as illustrated by arrow 110. This allows the catheter head 58 to scan a complete circumference of the vessel wall 24. In some further examples, the catheter head 58 is spun while being drawn-back, in direction 15, through the length of the portion of the vessel being analyzed.

However the spectra are resolved from the returning optical signals 102, the analyzer 42 makes an assessment of the state of the blood vessel wall 24 or other tissue of interest and, specifically area 22 that is opposite the catheter head 58, from collected spectra. The collected spectral response is used to determine whether the region of interest 22 of the blood vessel wall 24 comprises a lipid pool or lipid-rich atheroma, a disrupted plaque, a vulnerable plaque or thin-cap fibroatheroma (TCFA), a fibrotic lesion, a calcific lesion, and/or normal tissue in the current application. This categorized or even quantified information is provided to an operator via a user interface 70, or the raw discrimination or quantification results from the collected spectra are provided to the operator, who then makes the conclusion as to the state of the region of interest 22.

In one embodiment the information provided is in the form of a discrimination threshold that discriminates one classification group from all other spectral features. In another embodiment, the discrimination is between two or more classes from each other. In a further embodiment the information provided can be used to quantify the presence of one or more chemical constituents that comprises the spectral signatures of a normal or diseased blood vessel wall.

In therapeutic applications, the returning optical signals are used to control the therapy, such as the level and pulse period of a delivered beam, such as for ablation.

While this invention has been particularly shown and described with references to preferred embodiments thereof, it will be understood by those skilled in the art that various changes in form and details may be made therein without departing from the scope of the invention encompassed by the appended claims.

1. A multi fiber optic medical probe, comprising:
at least two optical fibers;
side-firing terminations for the at least two optical fibers; and
beam shaping apertures for controlling light propagating between the side-firing terminations and a region lateral to the probe.

2. A fiber optic medical probe as claimed in claim 1, wherein the at least two optical fibers comprise just two optical fibers.

3. A fiber optic medical probe as claimed in claim 1, wherein the at least two optical fibers comprises eight or more optical fibers.

4. A fiber optic medical probe as claimed in claim 1, wherein the at least two optical fibers comprise at least one single mode fiber and at least one multimode fiber.

5. A fiber optic medical probe as claimed in claim 1, wherein a core diameter of at least one of the optical fibers is less than about 10 micrometers and a core diameter of at least one other optical fiber of the at least two optical fibers is greater than 100 micrometers.

6. A fiber optic medical probe as claimed in claim 1, wherein the side firing terminations comprise angled endfaces for the at least two optical fibers.

7. A fiber optic medical probe as claimed in claim 6, wherein the angled endfaces are formed by polishing.

8. A fiber optic medical probe as claimed in claim 1, wherein the side firing terminations comprise at least one coreless block.

9. A fiber optic medical probe as claimed in claim 8, wherein the at least one coreless block comprises an angled endface.

10. A fiber optic medical probe as claimed in claim 9, wherein the at least one angled endface is formed by polishing.

11. A fiber optic medical probe as claimed in claim 9, wherein the at least one angled endface is metal coated.

12. A fiber optic medical probe as claimed in claim 8, wherein the at least one coreless block is attached to ends of the optical fibers.

13. A fiber optic medical probe as claimed in claim 8, wherein the at least one coreless block is fused to ends of the optical fibers.

14. A fiber optic medical probe as claimed in claim 1, further comprising at least one capillary tube over the side-firing terminations of the at least two optical fibers, at least one capillary tube providing the beam shaping apertures.

15. A fiber optic medical probe as claimed in claim 14, wherein the capillary tube comprises multiple bores for each of the at least two optical fibers.

16. A fiber optic medical probe as claimed in claim 1, further comprising capillary tubes over the side-firing terminations of the at least two optical fibers.

17. A fiber optic medical probe as claimed in claim 16, wherein the capillary tubes are attached to each other.

18. A fiber optic medical probe as claimed in claim 16, wherein the capillary tubes are bonded to each other.

19. A fiber optic medical probe as claimed in claim 16, further comprising a spacer block between the capillary tubes.

20. A fiber optic medical probe as claimed in claim 16, further comprising a wedge spacer between the capillary tubes for controlling an angle between the optical axes between the beam shaping apertures.

21. A fiber optic medical probe as claimed in claim 20, wherein the wedge spacer is integral with one of the capillary tubes.

22. A fiber optic medical probe as claimed in claim 1, wherein the beam shaping apertures are longitudinally offset along an axis of the probe with respect to each other.

23. A fiber optic medical probe as claimed in claim 1, further comprising at least three optical fibers, wherein one of the optical fiber is single mode fiber and other fibers are multimode fibers.

24. A method of gathering optical information using a medical probe, comprising:
transmitting an optical signal in a first optical fiber;
directing the optical signal to a region lateral to the probe with a side-firing termination to the first optical fiber;
controlling a beam shape of the optical signal;
collecting optical information with a second optical fiber and transmitting the optical information to an analyzer.

25. A method as claimed in claim 24, wherein the step of collecting optical information comprises collecting the optical information with multiple optical fibers.

26. A method as claimed in claim 24, wherein the step of transmitting the optical signal in the first optical fiber comprises transmitting the optical signal in single mode fiber.

27. A method as claimed in claim 24, wherein the step of collecting optical information comprises collecting the optical information with at least one multimode optical fiber.

28. A multi fiber optic medical probe, comprising:
at least one single mode optical fiber;
at least one multimode optical fiber;
side-firing terminations for the optical fibers having beam shaping apertures.

29. A fiber optic medical probe as claimed in claim 28, wherein the at least two optical fibers comprise just two optical fibers.

30. A fiber optic medical probe as claimed in claim 28, wherein the at least two optical fibers comprises eight or more optical fibers.

31. A fiber optic medical probe as claimed in claim 28, wherein a core diameter of the single mode optical fiber is less than about 10 micrometers and a core diameter of the multimode fiber is greater than 100 micrometers.

32. A fiber optic medical probe as claimed in claim 28, wherein the side firing terminations comprise angled endfaces for the at least two optical fibers.

33. A fiber optic medical probe as claimed in claim 32, wherein the angled endfaces are formed by polishing.

34. A fiber optic medical probe as claimed in claim 28, wherein the side firing terminations comprise at least one coreless block.

35. A fiber optic medical probe as claimed in claim 34, wherein the at least one coreless block comprises an angled endface.

36. A fiber optic medical probe as claimed in claim 35, wherein the at least one angled endface is formed by polishing.

37. A fiber optic medical probe as claimed in claim 36, wherein the at least one angled endface is coated.

38. A fiber optic medical probe as claimed in claim 34, wherein the at least one coreless block is attached to ends of the optical fibers.

39. A fiber optic medical probe as claimed in claim 34, wherein the at least one coreless block is fused to ends of the optical fibers.

40. A fiber optic medical probe as claimed in claim 28, further comprising at least one capillary tube over the side-firing terminations of the at least two optical fibers, at least one capillary tube providing the beam shaping apertures.

41. A fiber optic medical probe as claimed in claim 40, wherein the capillary tube comprises multiple bores for each of the at least two optical fibers.

42. A fiber optic medical probe as claimed in claim 28, further comprising capillary tubes over the side-firing terminations of the at least two optical fibers.

43. A fiber optic medical probe as claimed in claim 42, wherein the capillary tubes are attached to each other.

44. A fiber optic medical probe as claimed in claim 42, wherein the capillary tubes are bonded to each other.

45. A fiber optic medical probe as claimed in claim 42, further comprising a spacer block between the capillary tubes.

46. A fiber optic medical probe as claimed in claim 42, further comprising a wedge spacer between the capillary tubes for controlling an angle between the optical axes between the beam shaping apertures.

47. A fiber optic medical probe as claimed in claim 46, wherein the wedge spacer is integral with one of the capillary tubes.
